(19) United States
(12) Patent Application Publication
(10) Pub. No.: US 2022/0396811 A1
(43) Pub. Date: Dec. 15, 2022

(54) Systems and Devices for Delivering Cargo Material and Methods of Delivering Cargo Materials

(71) Applicant: Georgia Tech Research Corporation, Atlanta, GA (US)

(72) Inventor: Andrei Fedorov, Atlanta, GA (US)

(21) Appl. No.: 17/756,088

(22) PCT Filed: Nov. 23, 2020
(86) PCT No.: PCT/US2020/061798

§ 371 Date: May 17, 2022

Related U.S. Application Data
(60) Provisional application No. 62/940,553, filed on Nov. 26, 2019.

(51) Int. Cl. C12N 15/89; C12M 1/42; B01L 5/00

(52) U.S. Cl. CPC C12N 15/895; C12M 35/04; C12M 35/02; B01L 5/00; B01L 2400/0442

(57) ABSTRACT

The present disclosure provides for methods of delivering cargo material to a cell(s) in vivo or in vitro, devices configured to deliver cargo material to the cell, systems configured to deliver cargo material to the cell, and the like. The present disclosure can synergistically combine multiple technologies to improve the rate of cargo material introduction and utility of physical cellular modification in a transformative way compared to the current microinjection state-of-the art.

### CLAIM OF PRIORITY TO RELATED APPLICATION

This application claims priority to U.S. provisional application entitled “A Novel Method For Highly Localized, Versatile and Efficient Transfection Of Cells” having Ser. No.: 62/940,553 filed on Nov. 26, 2019, which is entirely incorporated herein by reference.

### STATEMENT REGARDING FEDERALLY SPONSORED RESEARCH OR DEVELOPMENT

This invention was made with government support under grants #1648035 awarded by the National Science Foundation (NSF) and DE-SC0010729 awarded by the Department of Energy (DOE). The government has certain rights in the invention.

### BACKGROUND

Modification of cells through the introduction of biomolecules has become a mainstream process for research and is gaining ground as an approach for treatment. For both in vivo and treatment-focused cellular modification, viral transfection is the main method employed, while for in vitro research-oriented modification, chemical (non-viral) transfection is more common. Both of these methods are “batch” methods which do not offer the ability to target a subset of cells for intentionally created heterogeneity. Furthermore, they are limited in which cargoes they can deliver efficiently. In contrast, physical delivery methods such as microinjection, in which a tiny hollow needle through which the cargo carrying liquid is injected pierces the cell membrane, allows precise control with almost no limits on cargo type, good delivery efficiency, and low toxicity, but at the cost of throughput and with the burden of requiring considerable skill. The recent use of automation has helped reduce the time required to locate cells and insert and withdraw the microneedle. Nevertheless, throughput and difficulty of implementation remain the main bottlenecks to greater adoption of microinjection.

### SUMMARY

Embodiments of the present disclosure provide for methods of delivering cargo material to a cell(s) in vivo or in vitro, devices configured to deliver cargo material to the cell, systems configured to deliver cargo material to the cell, and the like.

An embodiment of the present disclosure includes a method of delivery of a cargo to a cell, comprising: displacing a layer of fluid adjacent a membrane of the cell using a gas jet; directing a liquid beam at the membrane, wherein the liquid beam comprises at least a first cargo material, wherein the first cargo material is delivered into the cell.

Another embodiment of the present disclosure includes a system for delivery cargo material to a cell, comprising: a device configured to deliver to the cell a first cargo material, wherein a liquid beam ejector having a first end adjacent the cell directs the first cargo material at the cell using a liquid beam, wherein a liquid layer adjacent the cell is displaced using a gas jet delivered from a first end of a gas sheath, wherein the liquid beam ejector is configured to direct the liquid beam to the area of the cell having the liquid layer displaced.

Another embodiment of the present disclosure includes a device comprising: a liquid beam ejector configured to produce a liquid beam that exits a first end of the liquid beam ejector and a gas sheath is configured to produce a gas jet that exits a first end of the gas jet, wherein the liquid beam ejector and the gas sheath are configured to direct the liquid beam and gas jet, respectively, to a first area of a surface of a cell.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIGS. 1A and 1B illustrates cross-sectional views of an embodiment of the device that directs a liquid beam and a gas jet to a specific area.

FIGS. 2A and 2B illustrate cross-sectional views of an embodiment of the device and a cell in a fluid.

FIGS. 3A and 3B illustrates cross-sectional views of an embodiment of a device that directs a liquid beam and a gas jet to a specific area, where the liquid beam is coaxial with the gas jet.

FIG. 4 illustrates a cross-sectional view of an embodiment of the device and a cell in a fluid.

FIG. 5 illustrates a cross-sectional view of an embodiment of the device and a cell in a fluid.

FIGS. 6A and 6B illustrates cross-sectional views of an embodiment of the device that directs a liquid beam and a gas jet to a specific area.

FIGS. 7A and 7B illustrate cross-sectional views of an embodiment of the device and a cell in a fluid.

FIGS. 8A and 8B illustrates cross-sectional views of an embodiment of the device that directs a liquid beam and a gas jet to a specific area.

FIGS. 9A and 9B illustrate cross-sectional views of an embodiment of the device and a cell in a fluid.

FIGS. 10A and 10B illustrates cross-sectional views of an embodiment of the device that directs a liquid beam and a gas jet to a specific area.

FIGS. 11A and 11B illustrate cross-sectional views of an embodiment of the device and a cell in a fluid.

FIGS. 12A and 12B illustrates cross-sectional views of an embodiment of the device that directs a liquid beam and a gas jet to a specific area.

FIGS. 13A and 13B illustrate cross-sectional views of an embodiment of the device and a cell in a fluid.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of biochemistry, biology, cell biology, flow dynamics, analytical chemistry, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Discussion

Embodiments of the present disclosure provide for methods of delivering cargo material to a cell(s) in vivo or in vitro, devices configured to deliver cargo material to the cell, systems configured to deliver cargo material to the cell, and the like. Embodiments of the present disclosure can synergistically combine multiple technologies to improve the rate of cargo material introduction and utility of physical cellular modification in a transformative way compared to the current microinjection state-of-The art. The devices and systems of the present disclosure can be used to target applications requiring high levels of control, especially delivery to localized regions of tissue culture, while maintaining throughput and cargo material versatility as opposed to a focus on uniform modification of cell suspensions. The devices and systems of the present disclosure can be integrated with other medical devices such as surgical instruments, catheters, intravascular ultrasound and optical guiding probes and other systems used for medical treatment and diagnostics in vivo.

The present disclosure provides one or more advantages over other technologies such as microinjection. Embodiments of the present disclosure do not require direct contact, which eliminates several steps of the cargo material introduction process used by other techniques. Embodiments of the present disclosure can be designed to deliver cargo material much more quickly than other techniques. Operation in a “point-and-shoot” mode, rather than following the steps of grab-insert-inject-withdraw is also advantageous. As a result, embodiments of the present disclosure have increased rapidity and versatility relative to other techniques and permits simple, quick production of heterogeneously modified samples, including in cell and tissue cultures.

The devices and systems of the present disclosure allow for the precise control over the spatial distribution of cellular modification, with injection times 2-3 orders of magnitude faster than other techniques while also eliminating, microneedle insertion and retraction steps and providing for the versatility of “point and shoot” implementation. Embodiments of the present disclosure can transiently disrupt the cell membrane, enabling cargo material delivery to the cell interior. In general, a liquid jet including the cargo material is directed toward a cell in a fluid while also a gas jet or plasma gas jet (e.g., cold plasma) is directed to the same area of the cell. The gas jet will enable localized temporary drying, removal of thin liquid layers that could impede the liquid beam transport. In addition, the membrane can be porated, using one or a combination of techniques, using embodiments to allow for more efficient delivery of the cargo material.

The present disclosure provides for devices, systems, and methods for delivery of a cargo material to a cell, in vivo or in vitro. In an aspect, the present disclosure provides for delivery of a cargo material to a cell by displacing a layer of fluid adjacent a membrane of the cell using a gas jet. In an embodiment, the gas jet can be a plasma gas jet, where the plasma gas jet can also be used to porate the cell membrane. The cargo material can be delivered by directing a liquid beam at the exposed membrane, where the liquid beam comprises at least a first cargo material. In an aspect, the liquid beam can transiently disrupt and penetrate through a membrane of the cell.

The liquid beam can be a jet of fluid, a stream of droplets of fluid, or a combination thereof. The liquid beam can have a velocity of about 1 m/s to 100 m/s. The liquid beam (or droplets therein) can have a width or diameter of about 10 nm to 100 nm. The liquid beam can include one or more types of cargo material within water or a buffer solution. The first cargo material can include: nanoparticles (e.g., 5 nm to 50 nm), drug, molecules, imaging agents, peptides, antibodies, enzymes, viruses, proteins, RNA, DNA, macromolecular complexes, chromosomes, organelles, vesicles, xosomes, and combinations thereof. The cargo materials can be neutral or charged, positive or negative and/or can be influenced by magnetic fields based on the type of cargo material and. the desired way to deliver the material. The liquid beam can include more than one type of cargo material. Also, the type of cargo material delivered can be alternated during the course of delivery (e.g., switching between or amount different types of cargo materials during operation).

The liquid beam can be generated using a number of techniques or using a combination of techniques. In some embodiments, the liquid beam can be generated using a mechanically (e.g., forced pumping) generated liquid beam or hydrodynamic liquid beam, a magneto-phoretically generated liquid beam, an acousto-phoretically generated liquid beam, a thermo-phoretically generated liquid beam, and a combination thereof. The mechanically (via forced pumping) or hydrodynamic liquid beam generated liquid beam can be generated by a mechanical system. The magneto-phoretically generated liquid beam can be generated by a magnetic drive system. The acousto-phoretically generated liquid beam can be generated by an acoustic drive system. The thermo-phoretically generated liquid beam can be generated by a thermal drive system. In another embodiment, the liquid beam can be an electro-kinetically or electro-phoretically generated liquid beam. The electro-kinetically or electro-phoretically generated liquid beam can be generated by an electric drive system. One or more of these techniques can be combined to produce the liquid beam. Also, one or more of these techniques can be alternatively used to produce the liquid beam (e.g., mechanically generated liquid beam (e.g., neutral cargo materials) then switch to electro-kinetically or electro-phoretically generated liquid beam (e.g., charged cargo materials)), where for example, different types of cargo materials are delivered using the alternative techniques. The cargo material can be neutral, have net a positive charge, a net negative charge, be magnetic or be linked to a magnetic particle, encapsulated in solvophobic or solvophilic or amphiphilic (mixed phobic/philic) nanoparticle, or a combination thereof, so that the technique used to deliver the cargo material can be advantageously used. In addition, the technique used can be combined with the gas jet or plasma gas jet and/or the electrical system to produce the plasma gas jet to advantageously delivery the cargo material and/or porate the cell membrane.

In an example of combining the liquid beam generation and the plasma gas jet generation, the electro-kinetically or electro-phoretically generated liquid beam and the plasma gas jet are independently generated. In another aspect, the electro-kinetically or electro-phoretically generated liquid beam and the plasma gas jet are generated in electric and electronic communication with synchronization to one another. For example, the electro-kinetically or electro-phoretically generated liquid beam and the plasma gas jet, as well as the associated electronic components, can produce an applied electric field strength of the electrical system is above 1 kV/cm to less than 10 kV/cm or about 1 kV/cm to less than 9 kV/cm in a non-electrospray configuration or alternatively, can produce an applied electric field strength of the electric system is equal to or greater than 10 kV/cm to 50 kV/cm in an electrospray configuration. In an aspect, the electric system includes one more components (e.g., electric contacts) placed in particular places (e.g., at, on, or in electrical connection the liquid beam ejector, gas sheath, cell, fluid adjacent the cell, and the like and combinations thereof) as needed to generate the applied electric field strength.

The gas jet can be generated using a gas sheath that flows a gas at a flow rate of about 1 L/min to 10 L/min to displace the fluid disposed on and/or around the cell or tissue. The gas sheath is in gaseous communication with a gas system. The gas system flows the gas into the gas sheath to produce the gas jet, where the gas flow system is configured to operate in a continuous mode or pulsed mode. The gas comprising the gas jet (or plasma gas jet) can include air, oxygen, nitrogen, argon, helium, hydrogen, CO₂, H₂O, or a combination thereof (or plasma thereof or plasma of a combination thereof). The gas jet can be operated at a temperature of about 0 to 90° C. In one embodiment, the electric system on the gas sheath can be used to produce a plasma gas jet, where the gas jet flowing through the structure passes through the volume where the applied voltage (e.g., about 1 kV to 10 kV at frequency of 1 to 100 kHz) generates the plasma gas jet.

Cold atmospheric plasma (CAP) is an ionized gas operated at near-ambient temperatures. Cold plasmas, generated by continued or alternating discharge at low frequency (25-450 kHz), radio frequency (RF) (1-500 MHz), or microwave frequency (500 MHz to some GHz) at rather low pressure (10⁻²-10 Torr), are cold plasmas. Cold plasmas are characterized by energy and electronic density equal to 1-10 eV and 10¹⁰ cm⁻³, respectively. Their ionization degree is lower than 10⁻³ so that the gas phase is mainly made up of neutral species in an excited state (radicals). A characteristic of these plasmas is the absence of thermodynamic balance between the electronic temperature (several thousands of degrees) and that of the gas (near to the ambient). Cold plasmas are generally achieved by means of two types of energized structures: (1) a tubular-type reactor with an external coil or ring electrodes for excitation by RF discharge and (2) a bell-jar-type reactor with internal parallel-plate metallic electrodes. In the latter case, a low-frequency or RF voltage for discharge excitation is generally used. Microwave discharges are widely used as well, with various methods of application of microwave energy (the most frequent is the multimode cavity also called microwave oven mode). Magnetic fields could also be employed to assist the plasma mode: electron cyclotron resonance discharge and planar or cylindrical magnetrons. The electric system can be configured with the components to produce the desired plasma.

Atmospheric Pressure Plasma provides the highest possible plasma density. It is a unique, nonthermal glow discharge plasma operating at atmospheric pressure. The discharge uses a high flow rate of the feed gas including of primarily an inert carrier gas like He and a small amount of additive such as O₂, H₂O, CF₄, and the like, corona discharge and dielectric-barrier discharge (DBD) are some of the atmospheric pressure plasma sources. Corona Discharge is formed at atmospheric pressure by applying a low frequency or pulsed high voltage over an electrode pair. The electrodes have a large difference in size. This plasma includes a series of small lightning-type discharges.

The device and system can include a gas sheath and liquid beam ejector. The liquid beam ejector can include a capillary, a liquid wetted pillar configured to use the focused mechanical (oscillation), electric, acoustic, magnetic, and thermal fields to induce fluid motion along the pillar to produce the liquid beam, or a porous membrane configured to use the focused mechanical (oscillation), electric, acoustic, magnetic, and thermal fields, where each can generate the liquid beam.

The gas sheath and liquid beam ejector can be positioned above the fluid surrounding the cell or tissue at a first distance of about 10 micrometers to 5 millimeters. In operation, the gas jet (or plasma gas jet) displaces (e.g., pushes, evaporates, or both) the layer of fluid away from a first area of the cell so that the liquid beam impacts the membrane with a first velocity. If the fluid is not removed, the liquid beam may lose momentum or otherwise dissipate so that delivery of the cargo materials is not achieved. The displacement of the layer of fluid occurs in an area with an extent/diameter of about 10 micrometer to about 1 millimeter. The displacement of the layer of fluid can remove all of the fluid so that the membrane of the cell is nearly 100% exposed or a thin layer of fluid may remain, but is thin enough so the liquid beam, alone or in combination with other techniques to porate the cell membrane, is able to deliver the cargo material to the inside of the cell.

In another embodiment, the gas sheath and liquid beam ejector can be positioned within the fluid surrounding the cell or tissue and when in operation the displacement of the fluid forms a bubble within the layer of fluid adjacent the cell. The displacement can be caused by the gas jet or the plasma gas jet. The formation of the bubble pushes the layer of fluid away from a first area adjacent the cell so that the liquid beam impacts the membrane with a first velocity and delivers the cargo material. The bubble may also serve the function to assist in poration of the membrane due to the increase in pressure on the cell membrane as well as the shear stresses applied on the cell membrane due to gas flow in the bubble. Also, the plasma's charged species and free radicals (e.g., high concentrations of reactive oxygen and nitrogen species (RONS), including atomic nitrogen, oxygen, hydroxyl (OH⁻), singlet delta oxygen, superoxide, and nitric oxide NO) provide an additional mechanism for membrane transient disruption and pore formation for delivering cargo.

As described herein, the poration of the cell membrane can be accomplished using one or more techniques. The poration of the membrane can be caused by the plasma gas jet, the liquid beam, or a combination thereof. The plasma gas jet poration is plasma-induced poration. The poration caused by the liquid beam alone is mechanoporation, where a combination of the plasma gas jet and liquid beam causes electrohydrodynamic stress-induced poration (e.g., poration caused by both the electric force and mechanical shear if the impinging liquid beam is electrically charged). The liquid beam can transiently disrupt and penetrate through a membrane of the cell. In addition, the bubble within the layer of the fluid can porate the cell membrane using the mechanical pressure and shear as well as reaction with the reactive plasma species.

The membrane of the biological cells can be exposed to the gas jet or plasma gas jet for a certain time (e.g., from 1 second to 1 minute in one embodiment) that is optimal for membrane poration and maintaining cell viability.

The cell membrane can also be porated using electroporation. In particular, poration can be caused by one of more of the following: the electrically charged gas plasma, an electric field associated with a charged liquid beam impinging on the cell, an electric field due to an electric potential difference formed between a device electrode (e.g., used for charging the liquid beam or generating the gas plasma) and a counter-electrode, (e.g., where the counter electrode is connected to the substrate where the cells are placed or is incorporated into the cell layer), an electric field due to the potential difference between an electrode used for charging the liquid beam and an electrode used for plasma generation. For example, an applied electric field strength that can be applied using the liquid beam ejector (e.g., capillary) and/or gas sheath or independently of the liquid beam ejector and gas sheath. The applied electric field strength can be above 1 kV/cm to less than 10 kV/cm or about 1 kV/cm to less than 9 kV/cm or alternatively, can be equal to or greater than 10 kV/cm to 50 kV/cm. The electric system can be configured consistent with these embodiments as well as other configurations to achieve poration.

Embodiments of the method, as described above and herein, can be implemented using devices and systems as provided herein. In an embodiment, the device includes a liquid beam ejector configured to produce a liquid beam that exits a first end of the liquid beam ejector. The device also includes a gas sheath that is configured to produce a gas jet (or plasma gas jet) that exits a first end of the gas jet. The liquid beam ejector and the gas sheath are configured to direct the liquid beam and gas jet (or plasma gas het), respectively, to a first area of a surface of a cell.

In an embodiment, the capillary can be an inner capillary that is disposed coaxial within the gas sheath (outer gas sheath). In another embodiment, the capillary and the gas sheath are separate from one another and are not coaxial with one another.

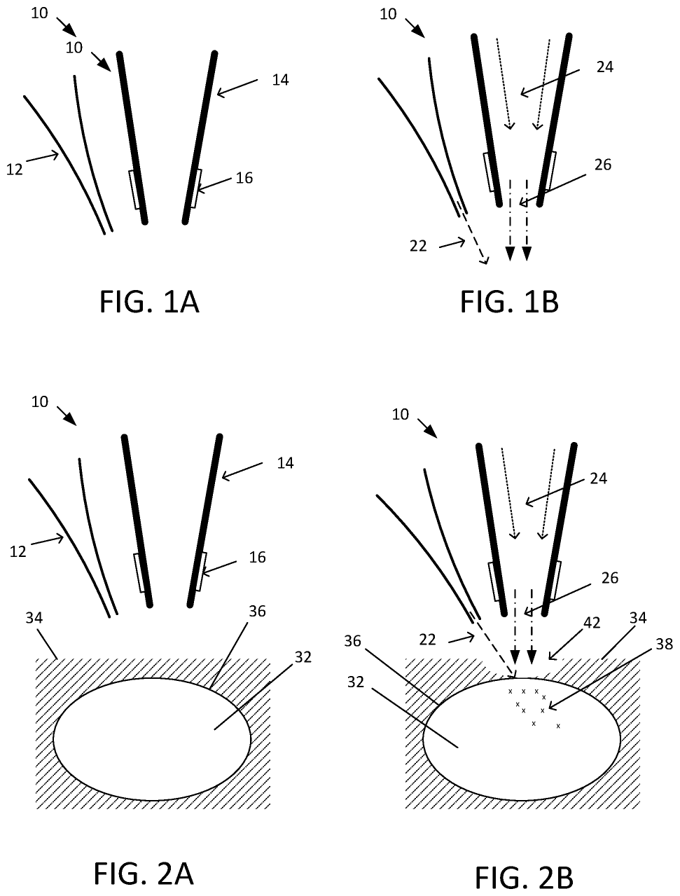

The liquid beam ejector (e.g., capillary) and the gas sheath can be electrically isolated or connected depending upon the desired design. Additional details are provided in FIGS. 1A-11B.

Embodiments of the device can be used in a system to deliver one or more types of cargo materials. The system can include a device configured to deliver to a cell a first cargo material. The system includes a liquid beam ejector such as a capillary having a first end adjacent the cell that directs the first cargo material at the cell using a liquid beam. A fluid layer adjacent the cell can be displaced using a gas jet delivered from a first end of a gas sheath, where the liquid beam ejector (e.g., capillary) is configured to direct the liquid beam to the area of the cell having the liquid layer displaced.

The liquid beam ejector is in fluidic communication with a fluid flow system, where the fluid flow system is configured to produce the liquid beam. The fluid flow system can include a mechanical system used to produce a hydrodynamic liquid beam, a magnetic drive system used to produce a magneto-phoretic liquid beam, an acoustic drive system used to produce an acousto-phoretic liquid beam, a thermal drive system used to produce a thermo-phoretic liquid beam, an electric drive system used to produce the electro-kinetic or electro-phoretic liquid beam, and a combination thereof. In addition, the fluid flow system can include an electrical system used to produce an electro-kinetic or electro-phoretic liquid beam. Two or more of these systems can be used together based on the design of the system, the desired outcome, and the cargo materials.

In an aspect, the mechanical system used to produce a hydrodynamic liquid beam can include a mechanical pump, such as a syringe pump, a gear pump, a peristaltic pump or to use a compressed gas for pumping the liquid.

In an aspect, the magnetic force system used to produce a magneto-phoretic liquid beam can include a system of electrodes that produce a magnetic field of desired magnitude and frequency/phase via electromagnetic induction phenomena and/or a set of permanent magnets of different polarity that impart a magnetic force on magnetic particles and cargos that incorporate the magnetized components (i.e., with non-zero magnetic susceptibility), resulting in their directed motion (pumping). The electrical and magnetic fields could be coupled to the nanostructures to focus the field along the nanostructure generating and amplifying the electromagnetic force (Maxwell stresses) to force the liquid flow along the nanostructure surface, producing the liquid beam emanating from the free end of the nanostructure.

In an aspect, the acoustic drive system used to produce an acousto-phoretic liquid beam can include a system of electromechanical transducers (e.g., piezoelectric or capacitive type) that generate the standing or traveling acoustic waves of desired amplitude and frequency that could apply the acoustic pressure gradient to the fluid and cargo in the fluid resulting in motion (pumping). The transducer could also be incorporated into the capillary tube and apply the squeezing action to force the liquid flow (pumping) similar to an inkjet printer. Alternatively, the acoustic fields could be coupled to the nanostructures to focus the field along the nanostructure generating and amplifying the acoustic surface wave to force the liquid flow along the nanostructure surface, producing the liquid beam emanating from the free end of the nanostructure.

In an aspect, the thermal drive system used to produce a thermo-phoretic liquid beam by applying the temperature gradient (e.g., using an array of heaters) to a fluid with cargo forcing it to move by thermophoretic force due to a Soret effect.

In an aspect, the electric drive system used to produce the electro-kinetic or electro-phoretic liquid beam can include one or more electrodes energized at different potential to produce the motion of charged particles and ions via electro-kinetic and electro-phoretic phenomena. It could also include the electrospray system that generates a jet/beam of liquid through the application of the Maxwell stresses at the free surface of the liquid at the tip of the capillary, for example.

The above listed techniques for producing the liquid beam could be used individually or in combination depending on the specific application requirements and the type of liquid/cargo that is being delivered. Other techniques capable of generating the liquid motion could also be used in addition to the examples provided in the preceding paragraphs.

The gas sheath is in gaseous communication with a gas system, where the gas system flow the gas into the gas sheath to produce the gas jet (e.g., continuous mode or transient pulsed mode). The gas flow system includes one or more flow controllers or valves interfaced with the gas source and the gas sheath. The gas sheath is electrically configured to produce a plasma gas jet at or near the first end of the gas sheath. When the fluid flow system is an electrical drive system, the liquid beam ejector (e.g., capillary) and the gas sheath are electrically isolated. In another embodiment, when the fluid flow system is an electrical drive system, the liquid beam ejector (e.g., capillary) and the gas sheath are electrically connected.

Now having described various embodiments, additional description is provided in FIGSs. 1A-5. FIGS. 1A and 1B illustrates cross-sectional views of a device 10 that directs a liquid beam and a gas jet to a specific area, for example at the surface of the membrane of a cell, to deliver a cargo material. The device 10 can be included in a system as described herein. While FIGS. 1A and 1B illustrate a single device, more than on device can be used (e.g., multiplexed) to deliver the same or different types of materials to the same cell or different cells, for example tissue or a plurality of cells suspended or in a fluid flow. The device 10 includes a capillary 12 and a gas sheath 14, and optionally an electric system 16 in communication with the gas sheath and/or the capillary (not shown). As depicted in FIGS. 1A and 1B, the capillary 12 and the gas sheath 14 are adjacent one another and facing a common area (not illustrated here). FIG. 1B illustrates the flow of the liquid beam 22 as it exits the capillary 12 and the gas jet 24 as it exits the gas sheath 14. In addition, FIG. 1B illustrates that a plasma gas jet 26 can be formed using the electric system 16, albeit this is shown in FIG. 1B, production of the plasma gas jet 26 is optional. The plasma gas jet 26 can be formed at or near the tip of the gas sheath 14 and extend out from the gas sheath 14.

FIGS. 2A and 2B illustrate cross-sectional views of the device 10 and a cell 32 in a fluid 34. The device 10 can be used in in vivo or in vitro. FIG. 2A illustrates the device 10 positioned a first distance from the cell 32 and the liquid 34. FIG. 2B illustrates the device 10 when in operation. In operation, the gas jet 24 or as shown, the plasma gas jet 26can be used to displace (see volume of displacement 42) the fluid 34 so that the surface of the membrane 36 of the cell 32 is exposed more directly to the liquid beam 22 so that the liquid beam 22 contacts little or no fluid 34, which might otherwise impede the liquid beam 22 directed at the membrane 36 surface. The liquid beam 22 includes at least one cargo material 38 that is to be delivered to the cell 32 through the membrane 36. One or more modes can be used to porate the membrane 36 to deliver the cargo material 38. One or more modes can be used to deliver different types of cargo material 38 to the cell 32. In one embodiment, the liquid beam 22 can deliver the cargo material 38 into the cell, while in another embodiment, the plasma gas jet 26, individually or in combination with the liquid beam 22, can be used to porate the membrane 36 and deliver the cargo material. The liquid beam 22 and/or the gas jet 24 and/or the plasma gas jet 26 can be used in a continuous mode or in a pulsed mode.

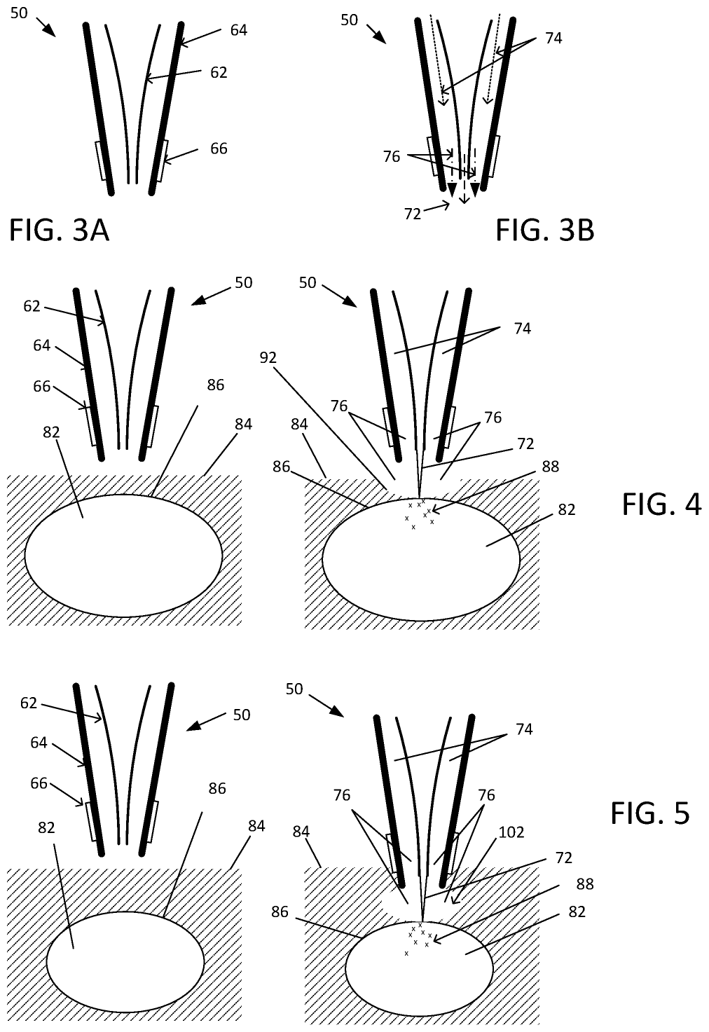

FIGS. 1A-2B illustrate the capillary 12 and the gas sheath 14 adjacent one another and this configuration can vary depending upon the desired set up. The distance between the capillary 12 and the gas sheath can vary from micrometers to millimeters as long as the liquid beam 22 and the gas jet 24 and/or the plasma gas jet 26 can be directed to the desired location (e.g., on the surface of a cell). FIGS. 3A-5 illustrate a configuration where the capillary 12 is positioned coaxially within the gas sheath.

FIGS. 3A and 3B illustrates cross-sectional views of a device 50 that directs a liquid beam and a gas jet to a specific area, where the liquid beam is coaxial with the gas jet. The device 50 can be included in a system as described herein. While FIGS. 3A and 3B illustrate a single device, more than on device can be used (e.g., multiplexed) to deliver the same or different types of materials to the same cell or different cells. The device 50 includes a capillary 62 (also referred to as “inner capillary”) that is positioned coaxially within a gas sheath 64 (also referred to as “outer gas sheath”). Optionally an electric system 66 in communication with the gas sheath and/or the capillary (not shown). As depicted in FIGS. 3A and 3B, the capillary 62 and the gas sheath 64 are coaxially positioned to direct a liquid beam 72 and gas jet 74 a common area (not illustrated here). FIG. 3B illustrates the flow of the liquid beam 72 as it exits the capillary 62 and the gas jet 74 as it exits the gas sheath 64, where the liquid beam 72 is coaxial with the gas jet 74. In addition, FIG. 3B illustrates that a plasma gas jet 76 can be formed using the electric system 66, albeit this is shown in FIG. 3B, production of the plasma gas jet 76 is optional. The plasma gas jet 76 can be formed at or near the tip of the gas sheath 64 and extend out from the gas sheath 64 and positioned coaxially with the liquid beam 72.

FIG. 4 illustrates a cross-sectional view of the device 50 and a cell 82 in a fluid 84. The device 50 can be used in in vivo or in vitro. FIG. 4 (left) illustrates the device 50 positioned a first distance from the cell 82 and the liquid 84. FIG. 4 (right) illustrates the device 50 when in operation. In operation, the gas jet 74 or as shown, the plasma gas jet 76 can be used to displace (see volume of displacement 92) the fluid 84 so that the surface of the membrane 86 of the cell 82 is exposed more directly to the liquid beam 72 so that the liquid beam 72 contacts little or no fluid 84, which might otherwise impede the liquid beam 72 directed at the membrane 86 surface. The liquid beam 72 includes at least one cargo material 88 that is to be delivered to the cell 82 through the membrane 86. One or more modes can be used to porate the membrane 86 to deliver the cargo material 88. One or more modes can be used to deliver different types of cargo material 88 to the cell 82. In one embodiment, the liquid beam 72 can deliver the cargo material 88 into the cell, while in another embodiment, the plasma gas jet 76, individually or in combination with the liquid beam 72, can be used to porate the membrane 86 and deliver the cargo material. The liquid beam 72 and/or the gas jet 74 and/or the plasma gas jet 76 can be used in a continuous mode or in a pulsed mode.

FIG. 5 illustrates a cross-sectional view of the device 50 and a cell 82 in a fluid 84. The device 50 can be used in in vivo or in vitro. FIG. 5 (left) illustrates the device 50 positioned a first distance from the cell 82 and the liquid 84. FIG. 5 (right) illustrates the device 50 when in operation positioned at a second distance from the cell 82 that has a portion of the device 50 within the fluid 84. In operation, the gas jet 74 or as shown, the plasma gas jet 76 can be used to displace the fluid 84 to form a bubble 102 so that the surface of the membrane 86 of the cell 82 is exposed more directly to the liquid beam 72 so that the liquid beam 72 contacts little or no fluid 84, which might otherwise impede the liquid beam 72 directed at the membrane 86 surface. The liquid beam 72 includes at least one cargo material 88 that is to be delivered to the cell 82 through the membrane 86. One or more modes can be used to porate the membrane 86 to deliver the cargo material 88. One or more modes can be used to deliver different types of cargo material 88 to the cell 82. In one embodiment, the liquid beam 72 can deliver the cargo material 88 into the cell, while in another embodiment, the plasma gas jet 76, individually or in combination with the liquid beam 72, can be used to porate the membrane 86 and deliver the cargo material 88. In another embodiment, the bubble 102 can produce a pressure that porates the cell 82, so that the bubble 102, plasma gas jet 76, and/or the liquid beam 72 can porate the cell membrane 86. The liquid beam 72 and/or the gas jet 74 and/or the plasma gas jet 76 can be used in a continuous mode or in a pulsed mode.

Now having described the devices 10 and 50 generally, additional details will be provided. The capillary 12 and 62 can be made of a material such as glass, silica, polymer or metal or combination of conductive and non-conductive materials for electrical isolation and have a length of about 1 cm to 10 cm. Along the length of the capillary 12 and 62 the capillary narrow to form a first end that can have an opening that has a longest inner dimension (e.g., inner width or inner diameter) of about 1 micrometer to 10 micrometers.

The capillary 12 and 62 is in fluidic communication with a fluid flow system, where the fluid flow system is configured to produce the liquid beam. The fluid flow system is in fluidic communication with the capillary and a source of the fluid (e.g., water, a buffer solution, cell growth media, organic solvents, and the like) and the cargo material. The fluid flow system can be a mechanical system used to produce a hydrodynamic liquid beam, a magnetic drive system used to produce a magneto-phoretic liquid beam, an acoustic drive system used to produce an acousto-phoretic liquid beam, a thermal drive system used to produce a thermo-phoretic liquid beam, an electric drive system used to produce the electro-kinetic or electro-phoretic liquid beam, or a combination thereof.

The fluid flow system can be an electrical system used to produce an electro-kinetic or electro-phoretic liquid beam. The fluid flow system and the capillary can operate in an electrospray configuration or a non-electrospray configuration. The fluid flow system and the capillary can operate to produce an applied electric field strength from above 1 kV/cm to less than 10 kV/cm (the non-electrospray configuration) or about 1 kV/cm to less than 9 kV/cm (the non-electrospray configuration). The fluid flow system and the capillary can operate to produce an applied electric field strength that is equal to or greater than 10 kV/cm to 50 kV/cm (electrospray configuration).

The gas sheath 14 and 64 can be made of a material such as glass, silica, metal or polymer and has a first end that has an opening that has a longest inner dimension (e.g., inner width or inner diameter) of about 5 micrometers to 100 micrometers. The gas sheath 14 and 64 and the capillary 12 and 62 can be electrically isolated or electrically connected.

The gas sheath and fluid flow system are the same or similar in FIGS. 1-5 and FIGS. 6A-11A so this description applies to both sets of figures.

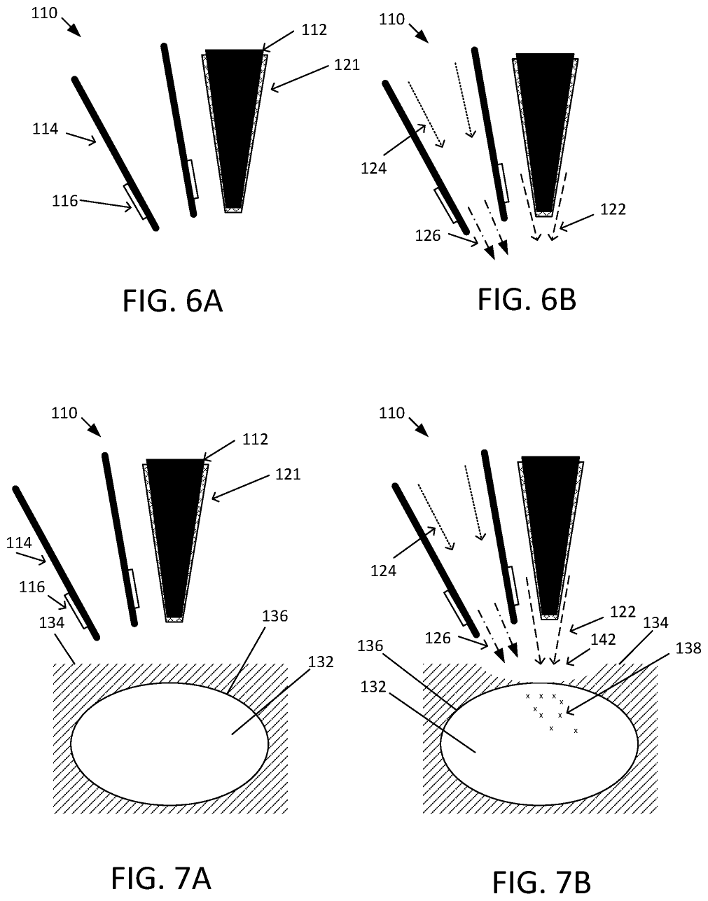

In FIGS. 6A-7B, the liquid beam ejector can be a pillar. While only a single pillar is illustrated, a plurality of pillars can be used in an array for a multiplex application. The liquid (e.g., including the cargo material) on the wetted pillar can be applied by dipping the pillar into a solution of liquid or have the liquid flowed or sprayed onto the pillar. The liquid beam can be produced by a liquid wetted pillar configured to use the focused mechanical (oscillation), electric, acoustic, magnetic, and thermal fields (such as those described in relation to the capillary or other embodiments described herein, which apply equally here) to induce fluid motion along the pillar to produce the liquid beam. The pillar can have a length of 100 nm to 100 μm, a width at half length of about 10 nm to 1 μm. The pillar can be made of a material selected from the group consisting of silicon, silica, glass, polymer and metal.

FIGS. 6A and 6B illustrates cross-sectional views of a device 110 that directs a liquid beam and a gas jet to a specific area, for example at the surface of the membrane of a cell, to deliver a cargo material. The device 110 can be included in a system as described herein. While FIGS. 6A and 6B illustrate a single device, more than on device can be used (e.g., multiplexed) to deliver the same or different types of materials to the same cell or different cells, for example tissue or a plurality of cells suspended or in a fluid flow. The device 110 includes a pillar 112 having a liquid 121 disposed on the surface and a gas sheath 114, and optionally an electric system 116 in communication with the gas sheath and/or the pillar (not shown). As depicted in FIGS. 6A and 6B, the pillar 112 and the gas sheath 114 are adjacent one another and facing a common area (not illustrated here). FIG. 6B illustrates the ejection of the liquid beam 122 and the gas jet 124 as it exits the gas sheath 114. In addition, FIG. 6B illustrates that a plasma gas jet 126 can be formed using the electric system 116, albeit this is shown in FIG. 6B, production of the plasma gas jet 126 is optional. The plasma gas jet 126 can be formed at or near the tip of the gas sheath 114 and extend out from the gas sheath 114.

FIGS. 7A and 7B illustrate cross-sectional views of the device 110 and a cell 132 in a fluid 134. The device 110 can be used in in vivo or in vitro. FIG. 7A illustrates the device 110 positioned a first distance from the cell 132 and the liquid 134. FIG. 7B illustrates the device 110 when in operation. In operation, the gas jet 124 or as shown, the plasma gas jet 126 can be used to displace (see volume of displacement 142) the fluid 134 so that the surface of the membrane 136 of the cell 132 is exposed more directly to the liquid beam 122 so that the liquid beam 122 contacts little or no fluid 134, which might otherwise impede the liquid beam 122 directed at the membrane 136 surface. The liquid beam 122 includes at least one cargo material 138 that is to be delivered to the cell 132 through the membrane 136. One or more modes can be used to porate the membrane 136 to deliver the cargo material 138. One or more modes can be used to deliver different types of cargo material 138 to the cell 132. In one embodiment, the liquid beam 122 can deliver the cargo material 138 into the cell, while in another embodiment, the plasma gas jet 126, individually or in combination with the liquid beam 122, can be used to porate the membrane 136 and deliver the cargo material. The liquid beam 122 and/or the gas jet 124 and/or the plasma gas jet 126 can be used in a continuous mode or in a pulsed mode.

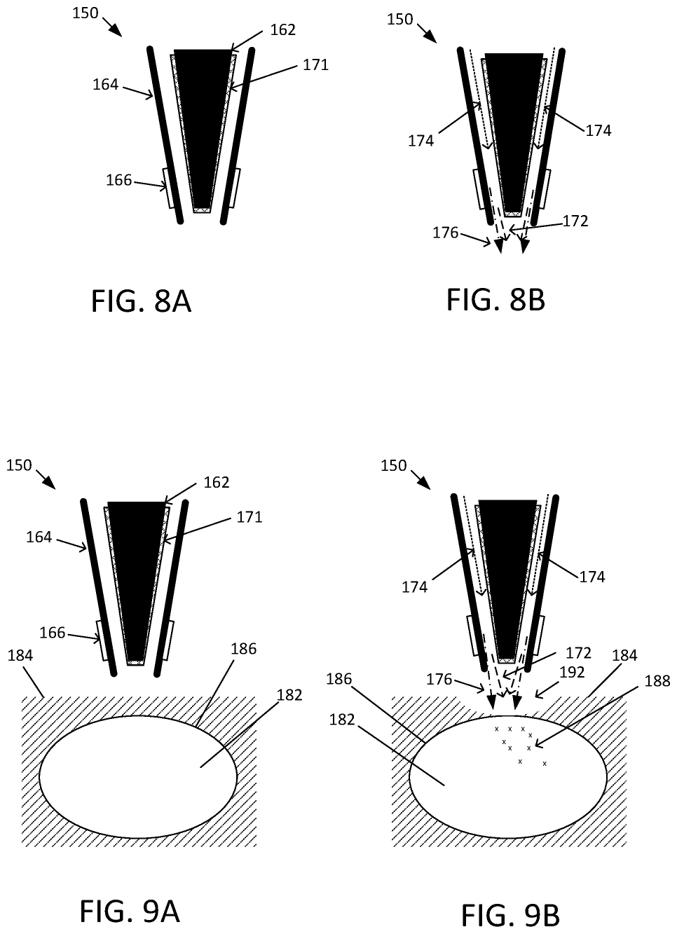

FIGS. 6A-7B illustrate the pillar 112 and the gas sheath 114 adjacent one another and this configuration can vary depending upon the desired set up. The distance between the pillar 112 and the gas sheath 114 can vary from micrometers to millimeters as long as the liquid beam 122 and the gas jet 124 and/or the plasma gas jet 126 can be directed to the desired location (e.g., on the surface of a cell). FIGS. 8A-9B illustrate a configuration where the pillar is positioned coaxially within the gas sheath.

FIGS. 8A and 8B illustrates cross-sectional views of a device 150 that directs a liquid beam and a gas jet to a specific area, where the liquid beam is coaxial with the gas jet. The device 150 can be included in a system as described herein. While FIGS. 8A and 8B illustrate a single device, more than on device can be used (e.g., multiplexed) to deliver the same or different types of materials to the same cell or different cells. The device 150 includes a pillar 162 having a fluid 171 disposed on the surface that is positioned coaxially within a gas sheath 164 (also referred to as “outer gas sheath”). Optionally an electric system 166 in communication with the gas sheath and/or the pillar (not shown). As depicted in FIGS. 8A and 8B, the pillar 162 and the gas sheath 164 are coaxially positioned to direct a liquid beam 172 and gas jet 174 a common area (not illustrated here). FIG. 8B illustrates the flow of the liquid beam 172 as it exits the pillar 162 and the gas jet 174 as it exits the gas sheath 174, where the liquid beam 172 is coaxial with the gas jet 174. In addition, FIG. 8B illustrates that a plasma gas jet 176 can be formed using the electric system 166, albeit this is shown in FIG. 8B, production of the plasma gas jet 176 is optional. The plasma gas jet 176 can be formed at or near the tip of the gas sheath 164 and extend out from the gas sheath 164 and positioned coaxially with the liquid beam 172.

FIGS. 9A and 9B illustrate cross-sectional views of the device 150 and a cell 182 in a fluid 184. The device 150 can be used in in vivo or in vitro. FIG. 9A illustrates the device 150 positioned a first distance from the cell 182 and the liquid 184. FIG. 9B illustrates the device 150 when in operation. In operation, the gas jet 174 or as shown, the plasma gas jet 176 can be used to displace (see volume of displacement 192) the fluid 184 so that the surface of the membrane 186 of the cell 182 is exposed more directly to the liquid beam 172 so that the liquid beam 172 contacts little or no fluid 184, which might otherwise impede the liquid beam 172 directed at the membrane 186 surface. The liquid beam 172 includes at least one cargo material 188 that is to be delivered to the cell 182 through the membrane 186. One or more modes can be used to porate the membrane 186 to deliver the cargo material 188. One or more modes can be used to deliver different types of cargo material 188 to the cell 182. In one embodiment, the liquid beam 172 can deliver the cargo material 188 into the cell, while in another embodiment, the plasma gas jet 176, individually or in combination with the liquid beam 172, can be used to porate the membrane 186 and deliver the cargo material. The liquid beam 172 and/or the gas jet 174 and/or the plasma gas jet 176 can be used in a continuous mode or in a pulsed mode.

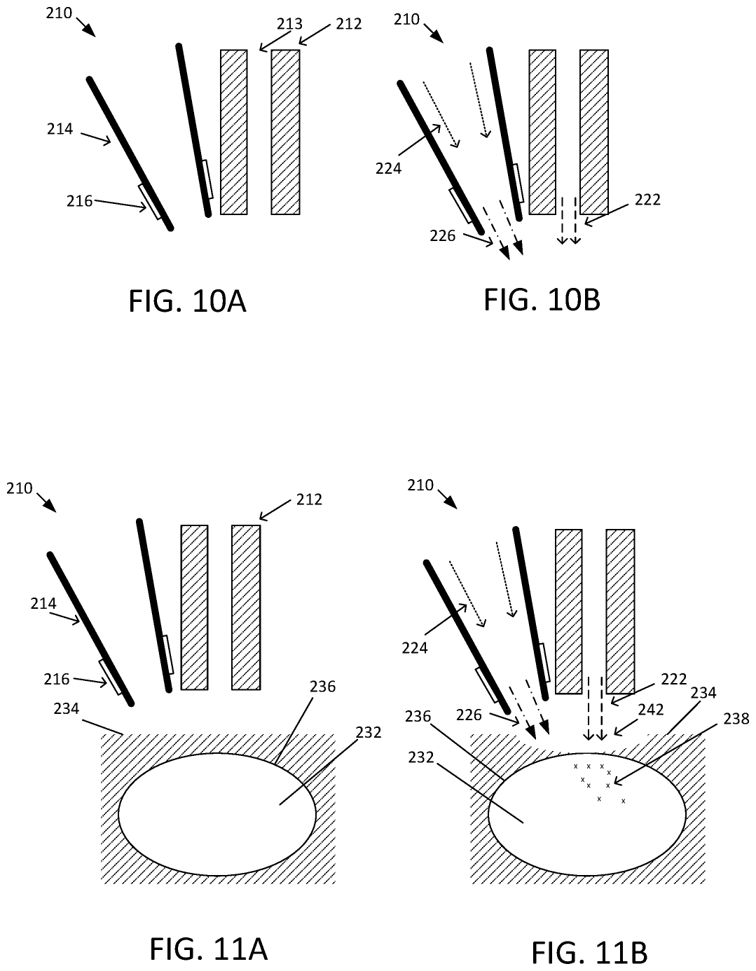

FIGS. 10A-13B illustrate that the liquid beam ejector can be a porous membrane. The porous membrane includes one or more pores. While only a single pore is illustrated, a plurality of pore can be used in an array for a multiplex application. The porous membrane is made of a material such as silicon, silica, alumina, polymer and metal. The pore can have a length of 1 μm to 1 mm and a width of about 10 nm to 1 μm and can be tubular. The porous membrane can be in fluidic communication with a fluid flow system, such as those described in relation to the capillary and those descriptions apply here equally. The liquid beam can be produced by a porous membrane to use the focused mechanical (oscillation), electric, acoustic, magnetic, and thermal fields (such as those described in relation to the capillary or other embodiments described herein, which apply equally here) to induce fluid motion along the pillar to produce the liquid beam.

FIGS. 10A and 10B illustrates cross-sectional views of a device 210 that directs a liquid beam and a gas jet to a specific area, for example at the surface of the membrane of a cell, to deliver a cargo material. The device 210 can be included in a system as described herein. While FIGS. 10A and 10B illustrate a single device, more than on device can be used (e.g., multiplexed) to deliver the same or different types of materials to the same cell or different cells, for example tissue or a plurality of cells suspended or in a fluid flow. The device 210 includes a porous membrane 212 having a pore 213 disposed and a gas sheath 214, and optionally an electric system 216 in communication with the gas sheath and/or the porous membrane (not shown). As depicted in FIGS. 10A and 10B, the porous membrane 212 and the gas sheath 214 are adjacent one another and facing a common area (not illustrated here). FIG. 10B illustrates the ejection of the liquid beam 222 and the gas jet 224 as it exits the gas sheath 214. In addition, FIG. 10B illustrates that a plasma gas jet 226 can be formed using the electric system 216, albeit this is shown in FIG. 10B, production of the plasma gas jet 226 is optional. The plasma gas jet 226 can be formed at or near the tip of the gas sheath 214 and extend out from the gas sheath 214.

FIGS. 11A and 11B illustrate cross-sectional views of the device 210 and a cell 232 in a fluid 234. The device 210 can be used in in vivo or in vitro. FIG. 11A illustrates the device 210 positioned a first distance from the cell 232 and the liquid 234. FIG. 11B illustrates the device 210 when in operation. In operation, the gas jet 224 or as shown, the plasma gas jet 2286 can be used to displace (see volume of displacement 242) the fluid 234 so that the surface of the membrane 236 of the cell 232 is exposed more directly to the liquid beam 222 so that the liquid beam 222 contacts little or no fluid 214, which might otherwise impede the liquid beam 222 directed at the membrane 236 surface. The liquid beam 222 includes at least one cargo material 238 that is to be delivered to the cell 232 through the membrane 236. One or more modes can be used to porate the membrane 236 to deliver the cargo material 238. One or more modes can be used to deliver different types of cargo material 238 to the cell 232. In one embodiment, the liquid beam 222 can deliver the cargo material 238 into the cell, while in another embodiment, the plasma gas jet 226, individually or in combination with the liquid beam 222, can be used to porate the membrane 236 and deliver the cargo material. The liquid beam 222 and/or the gas jet 224 and/or the plasma gas jet 226 can be used in a continuous mode or in a pulsed mode.

FIGS. 10A-11B illustrate the porous membrane 212 and the gas sheath 214 adjacent one another and this configuration can vary depending upon the desired set up. The distance between the porous membrane 212 and the gas sheath 214 can vary from micrometers to millimeters as long as the liquid beam 222 and the gas jet 224 and/or the plasma gas jet 226 can be directed to the desired location (e.g., on the surface of a cell). FIGS. 10A-11B illustrate a configuration where the porous membrane is positioned coaxially within the gas sheath.

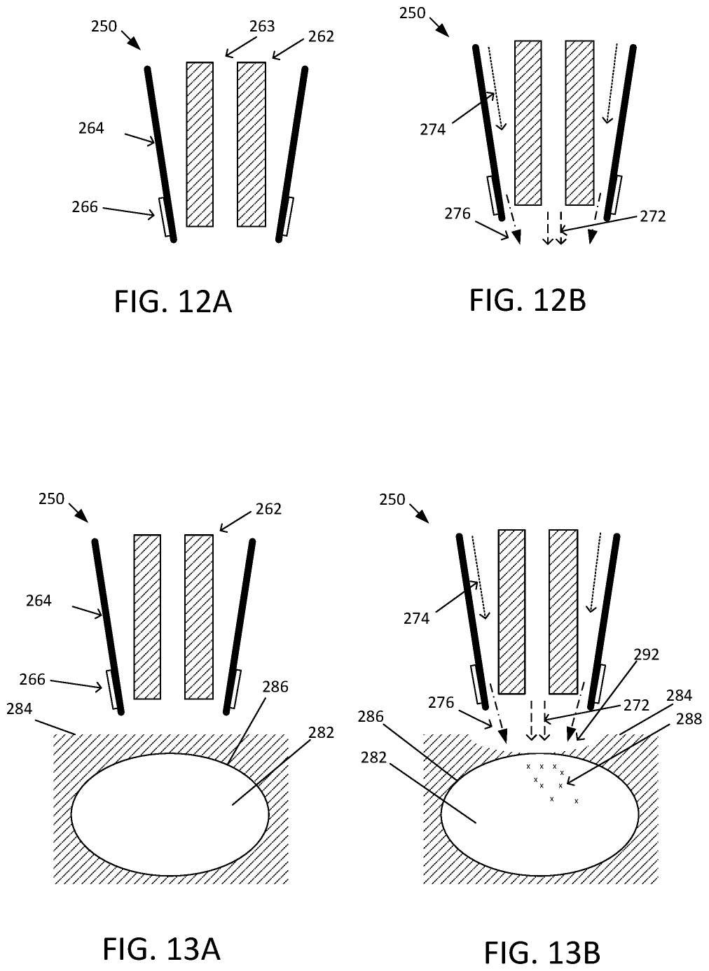

FIGS. 12A and 12B illustrates cross-sectional views of a device 250 that directs a liquid beam and a gas jet to a specific area, where the liquid beam is coaxial with the gas jet. The device 250 can be included in a system as described herein. While FIGS. 12A and 12B illustrate a single device, more than on device can be used (e.g., multiplexed) to deliver the same or different types of materials to the same cell or different cells. The device 250 includes a porous membrane 262 having a pore 263 that is positioned coaxially within a gas sheath 264 (also referred to as “outer gas sheath”). Optionally an electric system 266 in communication with the gas sheath and/or the pillar (not shown). As depicted in FIGS. 12A and 12B, the porous membrane 262 and the gas sheath 264 are coaxially positioned to direct a liquid beam 272 and gas jet 274 a common area (not illustrated here). FIG. 12B illustrates the flow of the liquid beam 272 as it exits the porous membrane 262 and the gas jet 274 as it exits the gas sheath 274, where the liquid beam 272 is coaxial with the gas jet 274. In addition, FIG. 12B illustrates that a plasma gas jet 276 can be formed using the electric system 266, albeit this is shown in FIG. 12B, production of the plasma gas jet 276 is optional. The plasma gas jet 276 can be formed at or near the tip of the gas sheath 264 and extend out from the gas sheath 264 and positioned coaxially with the liquid beam 272.

FIGS. 13A and 13B illustrate cross-sectional views of the device 250 and a cell 282 in a fluid 284. The device 250 can be used in in vivo or in vitro. FIG. 13A illustrates the device 250 positioned a first distance from the cell 282 and the liquid 284. FIG. 13B illustrates the device 250 when in operation. In operation, the gas jet 274 or as shown, the plasma gas jet 276 can be used to displace (see volume of displacement 292) the fluid 284 so that the surface of the membrane 286 of the cell 282 is exposed more directly to the liquid beam 272 so that the liquid beam 272 contacts little or no fluid 284, which might otherwise impede the liquid beam 272 directed at the membrane 286 surface. The liquid beam 272 includes at least one cargo material 288 that is to be delivered to the cell 282 through the membrane 286. One or more modes can be used to porate the membrane 286 to deliver the cargo material 288. One or more modes can be used to deliver different types of cargo material 288 to the cell 282. In one embodiment, the liquid beam 272 can deliver the cargo material 288 into the cell, while in another embodiment, the plasma gas jet 276, individually or in combination with the liquid beam 272, can be used to porate the membrane 286 and deliver the cargo material. The liquid beam 272 and/or the gas jet 274 and/or the plasma gas jet 276 can be used in a continuous mode or in a pulsed mode.

A specific embodiment will be described, but the present disclosure is not limited to this particular embodiment, rather this is presented for illustration purposes. In a particular aspect, the present disclosure provides for a method (and device and system to implement the method) for highly localized, versatile, efficient modification (e.g., delivery, transfection, and the like) of cells. The present disclosure allows for very precise control over the spatial distribution of cellular modification using a combination of physical delivery methods to transiently disrupt the cell membrane thus enabling cargo material delivery and to actively inject the cargo into the cell using the liquid beam produced by one of the described systems and methods for pumping a liquid with cargo. In one embodiment, the present disclosure uses a plasma gas jet as the primary permeabilization method augmented by electro-permeabilization and hydrodynamic shear force induced membrane disruption to allow an electric field induced liquid beam to directly transport cargo material (e.g., charged positive and/or negative) into a target cell, in vivo or in vitro. A tightly focused gas jet advects the plasma gas jet and delivers it to the target surface while also locally removing any interfering liquid surround the cell. The plasma destabilizes the cell membrane, The membrane destabilization is augmented by a high intensity electric field (e.g., 1 to 10 kV/cm) that has as its primary function formation of the cargo material carrying electrified liquid beam. The liquid beam, having a radius of about 100 nm, is produced via application of a strong DC pulse (e.g., 2 kV amplitude), and hydrodynamic impingement on the cell membrane completes membrane poration. Cargo material introduction is via a high velocity electro-kinetic flow, and with flow rates of about 1 nL/s, the total delivery time required is less than a millisecond. The utility of this synergetic approach to cargo material injection into cells will be increased due to its versatility and ability to perform “direct-write” cellular modification on demand. The present disclosure permits simple production of heterogeneously modified samples, including in vitro cell and tissue cultures, and also in vivo modification of accessible organ systems.

The present disclosure will be better understood upon reading the following numbered features, which should not be confused with the claims. Any of the numbered features below can, in some instances, be combined with features described elsewhere in this disclosure and such combinations are intended to form part of the disclosure and these combinations can be claimed.

Feature 1. A method of delivery of a cargo to a cell, comprising:

displacing a layer of fluid adjacent a membrane of the cell using a gas jet (optionally wherein the gas jet is a plasm gas jet, wherein the plasma gas jet porates a membrane of the cell);

directing a liquid beam at the membrane, wherein the liquid beam comprises at least a first cargo material, wherein the first cargo material is delivered into the cell (optionally wherein the liquid beam transiently disrupts and penetrates through a membrane of the cell).

Feature 2. The method of feature 1, wherein the liquid beam is a jet of fluid, a stream of droplets of fluid, or a combination thereof.
Feature 3. The method of any proceeding feature, wherein the liquid beam is an electro-kinetically or electro-phoretically generated liquid beam, wherein the first cargo material is charged.
Feature 4. The method of any proceeding feature, wherein the electro-kinetically or electro-phoretically generated liquid beam and the plasma gas jet are independently generated.
Feature 5. The method of any proceeding feature, wherein the electro-kinetically or electro-phoretically generated liquid beam and the plasma gas jet are generated in electric and electronic communication with synchronization to one another (optionally wherein an applied electric field strength of the electrical system is above 1 kV/cm to less than 10 kV/cm or about 1 kV/cm to less than 9 kV/cm, or optionally wherein an applied electric field strength of the electrical system is equal to or greater than 10 kV/cm to 50 kV/cm).
Feature 6. The method of any proceeding feature, wherein the liquid beam is selected from the group consisting of: an mechanically (via forced pumping) generated liquid beam, a magneto-phoretically generated liquid beam, an acousto-phoretically generated liquid beam, a thermo-phoretically generated liquid beam, and a combination thereof.
Feature 7. The method of any proceeding feature, wherein the liquid beam has a velocity of about 1 m/s to 100 m/s.
Feature 8. The method of any proceeding feature, wherein the liquid beam is about 10 nm to 100 nm in width (optionally, wherein the liquid beam droplets have a longest dimension of 10 to 100 nm).
Feature 9. The method of any proceeding feature, wherein the first cargo material is selected from the group consisting of: nanoparticles (e.g., 5 nm to 50 nm), drug molecules. imaging agents, peptides, antibodies, enzymes, viruses, proteins, RNA, DNA, macromolecular complexes, cichromosomes, organelles, vesicles, exosomes, and combinations thereof (optionally wherein each of these can he charged or neutral).
Feature 10. The method of any proceeding feature, wherein displacing includes positioning the device above the layer of fluid adjacent the cell by about 10 micrometers to 5 millimeters, wherein the gas jet pushes the layer of fluid away from a first area of the cell so that the liquid beam impacts the membrane with a first velocity.
Feature 11. The method of any proceeding feature, wherein displacing includes positioning the device within the layer of fluid adjacent the cell.
Feature 12. The method of any proceeding feature, wherein displacing includes forming a bubble within the layer of fluid adjacent the cell using a gas jet, wherein the gas jet pushes the layer of fluid away from a first area adjacent the cell to form the bubble so that the liquid beam impacts the membrane with a first velocity (optionally wherein the bubble causes poration of the membrane), (optionally wherein the gas jet is a plasma gas jet, wherein the plasma gas jet forms the bubble within the layer of the fluid adjacent the cell jet and displaces the layer of fluid away from a first area adjacent the cell so that the liquid beam impacts the membrane with a first velocity, optionally wherein the liquid beam transiently disrupts and penetrates through a membrane of the cell, optionally wherein the plasma gas jet porates the membrane).
Feature 13. The method of any proceeding feature, wherein the liquid beam is produced by a capillary, wherein a gas sheath is configured to produce the gas jet (optionally wherein the gas sheath is configured to produce the plasma gas jet).
Feature 14. The method of any proceeding feature, wherein the liquid beam is produced by a liquid wetted pillar configured to use the focused mechanical (oscillation), electric, acoustic, magnetic, and thermal fields to induce fluid motion along the pillar to produce the liquid beam, wherein a gas sheath is configured to produce the gas jet (optionally wherein the gas sheath is configured to produce the plasma gas jet) (wherein the liquid beam is generated by a liquid on an outer surface of pillar).
Feature 15. The method of any proceeding feature, wherein the liquid beam is produced by a porous membrane, wherein a gas sheath is configured to produce the gas jet (optionally wherein the gas sheath is configured to produce the plasma gas jet).
Feature 16. The method of any proceeding feature, wherein the delivery occurs in vivo.
Feature 17. The method of any proceeding feature, wherein the delivery occurs in vitro.
Feature 18. The method of any proceeding feature, further comprising poration of the membrane of the cell.
Feature 19. The method of any proceeding feature, wherein the poration of the membrane is electroporation of the membrane.
Feature 20. The method of any proceeding feature, wherein poration is caused by one of more of the following: an electrically charged gas plasma, an electric field associated with a charged liquid beam impinging on the cell, an electric field due to an electric potential difference formed between a device electrode (e.g., used for charging the liquid beam or generating the gas plasma) and a counter-electrode, (optionally where the counter electrode is connected to the substrate where the cells are placed or is incorporated into the cell layer), an electric field due to the potential difference between an electrode used for charging the liquid beam and an electrode used for plasma generation.
Feature 21. The method of any proceeding feature, wherein the electroporation is caused by an electric field strength locally (across the cell membrane) above 1 kV/cm to less than 10 kV/cm or about 1 kV/cm to less than 9 kV/cm or greater than 10 kV/cm to 50 kV/cm.
Feature 22. The method of any proceeding feature, wherein the poration of the membrane is caused by the plasma gas jet, the liquid beam, or a combination thereof (wherein the plasma gas jet poration is plasma-induced poration, wherein the poration caused by the liquid beam alone is mechanoporation, wherein a combination of the plasma gas jet and liquid beam causes electrohydrodynamic stress-induced poration (which is poration caused by both the electric force and mechanical shear if the impinging liquid beam is electrically charged).
Feature 23. A system for delivery cargo material to a cell, comprising:

a device configured to deliver to the cell a first cargo material, wherein a liquid beam ejector having a first end adjacent the cell directs the first cargo material at the cell using a liquid beam, wherein a liquid layer adjacent the cell is displaced using a gas jet delivered from a first end of a gas sheath, wherein the liquid beam ejector is configured to direct the liquid beam to the area of the cell having the liquid layer displaced.

Feature 24. The system of feature 23, wherein the liquid beam ejector comprises a capillary.
Feature 25. The system of any proceeding feature, wherein the liquid beam ejector comprises a pillar (wherein the pillar has a length of 100 nm to 100 μm, a width at half length of about 10 nm to 1 μm, wherein the pillar is made of a material selected from the group consisting of silicon, silica, glass, polymer and metal.
Feature 26. The system of any proceeding feature, wherein the liquid beam ejector comprises a porous membrane (wherein the porous membrane has at least one pore, wherein the pore has a length of 1 μm to 1 mm, a width of about 10 nm to 1 μm, wherein the porous membrane is made of a material selected from the group consisting of silicon, silica, alumina, polymer and metal, wherein the porous membrane is in fluidic communication with a fluid flow system).
Feature 27. The system of any proceeding feature, wherein the first end of the gas sheath has an opening having a longest inner dimension (e.g., inner width or inner diameter) of about 5 micrometers to 100 micrometers.
Feature 28. The system of any proceeding feature, wherein the first end of the capillary has an opening having a longest inner dimension (e.g., inner width or inner diameter) of about 1 micrometer to 10 micrometers.
Feature 29. The system of any proceeding feature, wherein the capillary is in fluidic communication with a fluid flow system, wherein the fluid flow system is configured to produce the liquid beam.
Feature 30. The system of any proceeding feature, wherein the fluid flow system is a selected from a mechanical system used to produce a hydrodynamic liquid beam, a magnetic drive system used to produce a magneto-phoretic liquid beam, an acoustic drive system used to produce an acousto-phoretic liquid beam, a thermal drive system used to produce a thermo-phoretic liquid beam, an electric drive system used to produce the electro-kinetic or electro-phoretic liquid beam, and a combination thereof.
Feature 31. The system of any proceeding feature, wherein the fluid flow system is an electrical system used to produce an electro-kinetic or electro-phoretic liquid beam (optionally wherein an applied electric field strength of the electrical system is above 1 kV/cm to less than 10 kV/cm or about 1 kV/cm to less than 9 kV/cm, or optionally wherein an applied electric field strength of the electrical system is equal to or greater than 10 kV/cm to 50 kV/cm).
Feature 32. The system of any proceeding feature, wherein the gas sheath is electrically configured to produce a plasma gas jet at or near the first end of the gas sheath.
Feature 33. The system of any proceeding feature, wherein when the fluid flow system is an electrical drive system, the capillary and the gas sheath are electrically isolated.
Feature 34. The system of any proceeding feature, wherein when the fluid flow system is an electrical drive system, the capillary and the gas sheath are electrically connected.
Feature 35. The system of any proceeding feature, wherein the gas sheath is in gaseous communication with a gas system, wherein the gas system flow the gas into the gas sheath to produce the gas jet (optionally wherein the gas flow system is configured to operate in a steady continuous mode or transient pulsed mode, optionally wherein the gas is selected from the group consisting of air, oxygen, nitrogen, argon, helium, hydrogen, CO₂, H₂O, or a combination thereof optionally wherein the gas jet is at a temperature of about 0 to 90° C.).
Feature 36. The system of any proceeding feature, wherein the capillary is an inner capillary having a first end adjacent the cell to direct the first cargo material at the cell using the liquid beam, wherein the gas sheath is an outer gas sheath, wherein the gas jet is delivered from a first end of the outer gas sheath that is coaxial with liquid beam exiting the inner capillary, where inner capillary is disposed coaxial within the outer gas sheath.
Feature 37. The system of any proceeding feature, wherein the capillary and the gas sheath are separate from one another and are not coaxial with one another.
Feature 38. A device comprising:

a liquid beam ejector configured to produce a liquid beam that exits a first end of the liquid beam ejector and a gas sheath is configured to produce a gas jet that exits a first end of the gas jet, wherein the liquid beam ejector and the gas sheath are configured to direct the liquid beam and gas jet, respectively, to a first area of a surface of a cell.

Feature 39. The system of feature 38, wherein the liquid beam ejector comprises a capillary.
Feature 40. The system of any proceeding feature, wherein the liquid beam ejector comprises a pillar (wherein the pillar has a length of 100 nm to 100 μm, a width at half length of about 10 nm to 1 μm, wherein the pillar is made of a material selected from the group consisting of silicon, silica, glass, polymer and metal).
Feature 41. The system of any proceeding feature, wherein the liquid beam ejector comprises a porous membrane (wherein the porous membrane has at least one pore, wherein the pore has a length of 1 μm to 1 mm, a width of about 10 nm to 1 μm, wherein the porous membrane is made of a material selected from the group consisting of silicon, silica, alumina, polymer and metal, wherein the porous membrane is in fluidic communication with a fluid flow system.
Feature 42. The device of any proceeding feature, wherein the gas sheath is electrically configured to produce a plasma at or near the first end of the gas sheath.
Feature 43. The device of any proceeding feature, wherein the capillary is an inner capillary, wherein the gas sheath is an outer gas sheath, where inner capillary is disposed coaxial within the outer gas sheath.
Feature 44. The device of any proceeding feature, wherein the capillary and the gas sheath are separate from one another and are not coaxial with one another.
Feature 45. The device of any proceeding feature, wherein the first end of the gas sheath has an opening having a longest inner dimension (e.g., inner width or inner diameter) of about 5 micrometers to 100 micrometers.
Feature 46. The device of any proceeding feature, wherein the first end of the capillary has an opening having a longest inner dimension (e.g., inner width or inner diameter) of about 1 micrometer to 10 micrometers.
Feature 47. The device of any proceeding feature, wherein the capillary is in fluidic communication with a fluid flow system, wherein the fluid flow system is configured to produce the liquid beam.
Feature 48. The device of any proceeding feature, wherein the fluid flow system is a selected from a mechanical system used to produce a hydrodynamic liquid beam, a magnetic drive system used to produce a magneto-phoretic liquid beam, an acoustic drive system used to produce an acousto-phoretic liquid beam, a thermal drive system used to produce a thermo-phoretic liquid beam, an electric drive system used to produce the electro-kinetic or electro-phoretic liquid beam, and a combination thereof.
Feature 49. The device of any proceeding feature, wherein the fluid flow system is an electrical system used to produce an electro-kinetic or electro-phoretic liquid beam (optionally wherein an applied electric field strength of the electrical system is from above 1 kV/cm to less than 10 kV/cm or about 1 kV/cm to less than 9 kV/cm, or optionally wherein an applied electric field strength of the electrical system is equal to or greater than 10 kV/cm to 50 kV/cm).
Feature 50. The device of any proceeding feature, wherein when the fluid flow system is an electrical drive system, wherein the capillary and the gas sheath are electrically isolated.
Feature 51. The device of any proceeding feature, wherein when the fluid flow system is an electrical drive system, wherein the capillary and the gas sheath are electrically connected.
Feature 52. The device of any proceeding feature, wherein the gas sheath is in gaseous communication with a gas system, wherein the gas system flows the gas into the gas sheath to produce the gas jet (optionally wherein the gas flow system is configured to operate in an continuous mode or pulsed mode, optionally wherein the gas is selected from the group consisting of air, oxygen, nitrogen, argon, helium, hydrogen, CO₂, H₂O, or a combination thereof, optionally wherein the gas jet is at a temperature of about 0 to 90° C.).

Ratios, concentrations, amounts, and other numerical data may be expressed in a range format. It is to be understood that such a range format is used for convenience and brevity, and should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1% to about 5%, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figure of the numerical value. In addition, the phrase “about ‘x’ to ‘y’” includes “about ‘x’ to about ‘y’”.

Unless defined otherwise, all technical and scientific terms used have the same meaning as commonly understood by one of ordinary skill in the art to which this disclosure belongs. Although any methods and materials similar or equivalent to those described can also be used in the practice or testing of the present disclosure, the preferred methods and materials are now described.

Embodiments of the present disclosure will employ, unless otherwise indicated, techniques of separating, testing, and constructing materials, which are within the skill of the art. Such techniques are explained fully in the literature.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

1. A method of delivery of a cargo to a cell, comprising:
displacing a layer of fluid adjacent a membrane of the cell using a gas jet;
directing a liquid beam at the membrane, wherein the liquid beam comprises at least a first cargo material, wherein the first cargo material is delivered into the cell.

2. The method of claim 1, wherein the liquid beam is a jet of fluid, a stream of droplets of fluid, or a combination thereof.

3. The method of claim 1, wherein the liquid beam is selected from the group consisting of: a mechanically generated liquid beam, a magneto-phoretically generated liquid beam, an acousto-phoretically generated liquid beam, a thermo-phoretically generated liquid beam, and a combination thereof.

4. The method of claim 1, wherein the liquid beam has a velocity of about 1 m/s to 100 m/s and wherein the liquid beam is about 10 nm to 100 nm in width or wherein the liquid beam droplets have a longest dimension of 10 to 100 nm.

5. The method of claim 1, wherein the first cargo material is selected from the group consisting of: nanoparticles, drug molecules, imaging agents, peptides, antibodies, enzymes. viruses, proteins, RNA, DNA, macromolecular complexes, chromosomes, organelles, vesicles, exosomes, and combinations thereof, wherein each of these is neutral or charged.

6. The method of claim 1, wherein displacing includes positioning the device above the layer of fluid adjacent the cell by about 10 micrometers to 5 millimeters, wherein the gas jet pushes the layer of fluid away from a first area of the cell so that the liquid beam impacts the membrane with a first velocity.

7. The method of claim 1, wherein displacing includes positioning the device within the layer of fluid adjacent the cell or wherein displacing includes forming a bubble within the layer of fluid adjacent the cell using a gas jet, wherein the gas jet pushes the layer of fluid away from a first area adjacent the cell to form the bubble so that the liquid beam impacts the membrane with a first velocity.

8-11. (canceled)

12. The method of claim 11, wherein the poration of the membrane is electroporation of the membrane.

13-14. (canceled)

15. The method of claim 1, wherein the poration of the membrane is caused by the plasma gas jet, the liquid beam, or a combination thereof.

16. A system for delivery cargo material to a cell, comprising:
a device configured to deliver to the cell a first cargo material, wherein a liquid beam ejector having a first end adjacent the cell directs the first cargo material at the cell using a liquid beam, wherein a liquid layer adjacent the cell is displaced using a gas jet delivered from a first end of a gas sheath, wherein the liquid beam ejector is configured to direct the liquid beam to the area of the cell having the liquid layer displaced.

17. The system of claim 16, wherein the liquid beam is produced by a capillary, a liquid wetted pillar, or a porous membrane.

18. The system of claim 16, wherein the capillary is in fluidic communication with a fluid flow system, wherein the fluid flow system is configured to produce the liquid beam.

19. The system of claim 18, wherein the fluid flow system is a selected from a mechanical system used to produce a hydrodynamic liquid beam, a magnetic drive system used to produce a magneto-phoretic liquid beam, an acoustic drive system used to produce an acousto-phoretic liquid beam, a thermal drive system used to produce a thermo-phoretic liquid beam, an electric drive system used to produce the electro-kinetic or electro-phoretic liquid beam, and a combination thereof.

20. (canceled)

21. The system of claim 16, wherein the gas sheath is electrically configured to produce a plasma gas jet at or near the first end of the gas sheath.

22. The system of claim 16, wherein when the fluid flow system is an electrical drive system, the capillary and the gas sheath are electrically isolated.

23. The system of claim 16, wherein when the fluid flow system is an electrical drive system, the capillary and the gas sheath are electrically connected.

24. The system of claim 16, wherein the gas sheath is in gaseous communication with a gas system, wherein the gas system flow the gas into the gas sheath to produce the gas jet.

25. The system of claim 16, wherein the capillary is an inner capillary having a first end adjacent the cell to direct the first cargo material at the cell using the liquid beam, wherein the gas sheath is an outer gas sheath, wherein the gas jet is delivered from a first end of the outer gas sheath that is coaxial with liquid beam exiting the inner capillary, where inner capillary is disposed coaxial within the outer gas sheath.

26. A device comprising:
a liquid beam ejector configured to produce a liquid beam that exits a first end of the liquid beam ejector and a gas sheath is configured to produce a gas jet that exits a first end of the gas jet, wherein the liquid beam ejector and the gas sheath are configured to direct the liquid beam and gas jet, respectively, to a first area of a surface of a cell.

27. The device of claim 26, wherein the gas sheath is electrically configured to produce a plasma at or near the first end of the gas sheath.

28-30. (canceled)
